(19) United States
(12) Patent Application Publication
(10) Pub. No.: US 2025/0034068 A1
(43) Pub. Date: Jan. 30, 2025

(54) PURIFICATION PROCESS OF ORGANIC SOLVENT AND DEHYDRATION SYSTEM

(71) Applicant: DONGWOO FINE-CHEM CO., LTD., Iksan-si (KR)

(72) Inventors: Hwan-Min KIM, Iksan-si (KR); Dong-Seok SEO, Iksan-si (KR); Uk LEE, Iksan-si (KR)

(73) Assignee: DONGWOO FINE-CHEM CO., LTD., Iksan-si (KR)

(21) Appl. No.: 18/785,887

(22) Filed: Jul. 26, 2024

(30) Foreign Application Priority Data
Jul. 27, 2023 (KR) 10-2023-0098006

(51) Int. Cl. C07C 29/80

(52) U.S. Cl. CPC C07C 29/80

(57) ABSTRACT

The present disclosure relates to an organic solvent purification process and a dehydration system for obtaining a high-purity organic solvent, and according to the present disclosure, a high-purity organic solvent may be obtained by removing an inflow of impurities during a dehydration process.

### TECHNICAL FIELD

The present disclosure relates to a purification process of an organic solvent, and a dehydration system.

### BACKGROUND ART

Organic solvents used as process solutions and/or cleaning solutions in a semiconductor process are managed to have high purity by controlling the total amount of impurities, and an increase in the content of the impurities may lead to substrate contamination, process failure, wafer defects and the like. Specifically, when an organic solvent includes the impurities, errors occur during pattern formation, or adhesion of a deposited thin film is inhibited. In addition, there is a possibility that residual organic contaminants may react with a silicon substrate during a thermal process to form compounds such as silicon carbide. This may cause damage to electrical performance of a semiconductor circuit, which may ultimately affect stability of performance of a device including the same and overall productivity.

In particular, isopropyl alcohol is widely used for purposes such as cleaning and drying in a semiconductor device manufacturing process. With its superior solvent properties, isopropyl alcohol is widely used as a cleaning solution for IT components such as semiconductors and LCDs, and high-purity isopropyl alcohol is required for wafer cleaning in a semiconductor manufacturing process. In order to obtain high-purity isopropyl alcohol, a process of removing moisture in the organic solvent is necessary, and a method of removing moisture in an organic solvent using a molecular sieve such as zeolite has been used in the art as disclosed in Korean Patent Application Laid-Open No. 2014-0032331. However, using a molecular sieve included for organic solvent dehydration may reduce cleaning performance of a cleaning solution since metallic impurities such as boron remain in a purified organic solvent, and particularly in the case of a semiconductor cleaning solution, there is a problem of causing wafer defects. Accordingly, an organic solvent purification process and a dehydration system capable of efficient dehydration without remaining impurities have been required.

### PRIOR ART DOCUMENTS

### Patent Documents

(Patent Document 1] Korean Patent Application Laid-Open No. 10-2014-0032331

### DISCLOSURE

### Technical Problem

The present disclosure has been made in view of the above, and is directed to providing a purification process of an organic solvent capable of obtaining a high-purity organic solvent by removing impurities included in a molecular sieve, which is a dehydration means, through a pretreatment process.

In addition, the present disclosure is directed to providing a dehydration system capable of obtaining a high-purity organic solvent by using a molecular sieve that has undergone a pretreatment process.

However, objects to be addressed by the present disclosure are not limited to the objects mentioned above, and other objects not mentioned will be clearly appreciated by those skilled in the art from the following description.

### Technical Solution

One embodiment of the present disclosure provides a purification process of an organic solvent, the process including: removing moisture of an organic solvent using a dehydration means; and distilling the moisture-removed organic solvent, wherein the dehydration means includes a pretreated molecular sieve, and the pretreatment is immersing the molecular sieve in the organic solvent.

In the present disclosure, the organic solvent may be an alcohol-based organic solvent.

In the present disclosure, the organic solvent may be one or more selected from among isopropyl alcohol, propyl alcohol, butyl alcohol and hexyl alcohol.

In the present disclosure, the molecular sieve may be zeolite.

In the present disclosure, the molecular sieve may have a pore size of 3 Å to 5 Å.

In the present disclosure, the pretreatment may include immersing the molecular sieve in the organic solvent for 180 days or longer.

In the present disclosure, a content of boron included in the organic solvent purified according to the organic solvent purification process may be 1 ppb or less.

In the present disclosure, the purification process of an organic solvent may further include storing the purified organic solvent at a temperature of 30° C. or lower.

In addition, one embodiment of the present disclosure provides a dehydration system for removing water from an organic solvent, the system including: a first dry bed unit and a second dry bed unit; a first pipe unit connected to each surface of the first dry bed unit and the second dry bed unit and combined into one pipe so as to introduce or release an organic solvent to the first dry bed unit and the second dry bed unit; a second pipe unit connected to each surface of the first dry bed unit and the second dry bed unit and combined into one pipe so as to supply the organic solvent from the first dry bed unit and the second dry bed unit to a distillation column; and a first pretreatment solvent discharge unit connected to one surface of the first pipe unit so as to discharge the organic solvent supplied to the first dry bed unit and a second pretreatment solvent discharge unit connected to one surface of the first pipe unit so as to discharge the organic solvent supplied to the second dry bed unit.

In the present disclosure, the first pipe unit may include a first control valve capable of adjusting a flow rate of the organic solvent introduced to or released from the first dry bed unit and a second control valve capable of adjusting a flow rate of the organic solvent introduced to or released from the second dry bed unit, the second pipe unit may include a first release valve and a second release valve capable of adjusting a flow rate of the organic solvent supplied from the first dry bed unit and the second dry bed unit to the distillation column, and the first pretreatment solvent discharge unit and the second pretreatment solvent discharge unit may respectively include a first discharge valve and a second discharge valve capable of adjusting a flow rate of the discharged organic solvent.

In the present disclosure, the dehydration system may further include a third dry bed unit and a fourth dry bed unit.

In the present disclosure, when any one of the first dry bed unit and the second dry bed unit is driven to remove water from the organic solvent, the third dry bed unit and the fourth dry bed unit may be driven to pretreat a dehydration means.

In addition, one embodiment of the present disclosure provides a purification process of an organic solvent, the process including the dehydration system.

### Advantageous Effects

When an organic solvent is purified according to a purification process of the present disclosure, a content of impurities that can be included in a pretreatment process is reduced, and a high-purity organic solvent can be obtained.

In addition, when water is removed from an organic solvent through a dehydration system of the present disclosure, a content of impurities that can be included in a pretreatment process is reduced, and a high-purity organic solvent can be obtained.

### BRIEF DESCRIPTION OF DRAWINGS

FIG. 1 is a diagram illustrating an overall process including an organic solvent purification process of one embodiment of the present disclosure.

FIG. 2 is a diagram illustrating one example of a dry bed unit 111 of the present disclosure.

FIG. 3 is a diagram illustrating another example of a dry bed unit 111 of the present disclosure.

FIG. 4 is a diagram illustrating one example of a dry bed unit 111 in which a mesh is installed on an inlet and an outlet.

### BEST MODE

The present disclosure relates to an organic solvent purification process and a dehydration system for obtaining a high-purity organic solvent, and an organic solvent purified thereby. In addition, the present disclosure includes a storage process of the purified organic solvent.

More specifically, the present disclosure relates to a purification process of an organic solvent, the process including: removing moisture of an organic solvent using a dehydration means; and distilling the moisture-removed organic solvent, wherein the dehydration means includes a pretreated molecular sieve, and the pretreatment is immersing the molecular sieve in the organic solvent.

The present disclosure has an advantage of obtaining a high-purity organic solvent by using the pretreated molecular sieve as a dehydration means to reduce a content of impurities that may be included in the pretreatment process.

In addition, the present disclosure relates to a dehydration system for obtaining a high-purity organic solvent.

More specifically, the present disclosure relates to a dehydration system including: a first dry bed unit 200-1 and a second dry bed unit 200-2; a first pipe unit 101 connected to each surface of the first dry bed unit 200-1 and the second dry bed unit 200-2 and combined into one pipe so as to introduce or release an organic solvent to the first dry bed unit 200-1 and the second dry bed unit 200-2; a second pipe unit 102 connected to each surface of the first dry bed unit 200-1 and the second dry bed unit 200-2 and combined into one pipe so as to supply the organic solvent from the first dry bed unit 200-2 and the second dry bed unit 200-2 to a distillation column 120; and a first pretreatment solvent discharge unit 103-1 connected to one surface of the first pipe unit so as to discharge the organic solvent supplied to the first dry bed unit 200-1 and a second pretreatment solvent discharge unit 103-2 connected to one surface of the first pipe unit so as to discharge the organic solvent supplied to the second dry bed unit 200-2.

Hereinafter, preferred embodiments of the present disclosure will be described in detail. However, these embodiments are presented for illustrative purposes only in order to more specifically describe the present disclosure, and it will be obvious to those skilled in the art that the scope of the present disclosure is not limited by these embodiments.

The terms ‘include (comprise)’ and/or ‘including (comprising)’ used in the present specification are used in a sense of not excluding the presence or addition of one or more other constituents, steps, operations and/or constituents in addition to the mentioned constituents, steps, operations and/or the constituents.

The term ‘purification’ used in the present specification means removing impurities to purify a substance, and does not mean removing 100% of impurities only, and may mean removing impurities so that the purity is 90% by weight or greater, preferably 95% by weight or greater, more preferably 99% by weight or greater, and most preferably 99.999%% by weight. In the present disclosure, a ‘purified organic solvent’ may mean a state in which organic impurities and/or metal-based impurities are removed.

The term ‘dehydration’ used in the present specification means removing moisture from an organic solvent including moisture, and does not mean removing 100% only, and may mean that moisture included in an organic solvent is included in an amount of 9% by weight or less, preferably 5% by weight or less, more preferably 1% by weight or less, and most preferably 30 ppm or less. In the present disclosure, a ‘dehydrated organic solvent’ may mean a state in which moisture is removed from an organic solvent.

The term ‘immersion’ used in the present specification may mean not only an act of soaking in a solvent as a dictionary meaning, but also an act of permeating an organic solvent into a molecular sieve using a mechanical means or method. Examples of the mechanical means or method may include mixing, stirring, solvent circulation or ultrasonic waves, however, the means or method is not particularly limited as long as it is for permeating an organic solvent into the molecular sieve, and methods known in the art may be used without limit.

### <Purification Process of Organic Solvent>

The organic solvent purification process of the present disclosure includes: removing moisture of an organic solvent using a dehydration means; and distilling the moisture-removed organic solvent. In particular, the dehydration means includes a pretreatment molecular sieve in the present disclosure, and the pretreatment is immersing the molecular sieve in the organic solvent. By performing a dehydration process through such a pretreated molecular sieve in the organic solvent purification process of the present disclosure, a content of impurities that may be included in the pretreatment process, particularly, boron, is significantly reduced, and a high-purity organic solvent may be obtained.

### Organic Solvent

The organic solvent of the present disclosure may function as an immersion solvent used for pretreatment of a molecular sieve while being a solvent to be purified. Specifically, as the immersion solvent, it is preferred to use the same type of organic solvent as the organic solvent to be purified. When pretreatment of a molecular sieve is performed by immersing in a different type of organic solvent from the organic solvent to be purified, the immersion solvent becomes an impurity to the organic solvent to be purified, and it may be difficult to obtain a high-purity organic solvent.

Examples of the organic solvent may include methanol, ethanol, 1-propanol, isopropyl alcohol (isopropanol), n-propanol, 2-methyl-1-propanol, n-butanol, 2-butanol, tert-butanol, 1-pentanol, 2-pentanol, 3-pentanol, n-hexanol, cyclohexanol, 2-methyl-2-butanol, 3-methyl-2-butanol, 2-methyl-1-butanol, 3-methyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 2-ethyl-1-butanol, 2,2-dimethyl-3-pentanol, 2,3-dimethyl-3-pentanol, 2,4-dimethyl-3-pentanol, 4,4-dimethyl-2-pentanol, 3-ethyl-3-heptanol, 1-heptanol, 2-heptanol, 3-heptanol, 2-methyl-2-hexanol, 2-methyl-3-hexanol, 5-methyl-1-hexanol, 5-methyl-2-hexanol, 2-ethyl-1-hexanol, methylcyclohexanol, trimethylcyclohexanol, 4-methyl-3-heptanol, 6-methyl-2-heptanol, 1-octanol, 2-octanol, 3-octanol, 2-propyl-1-pentanol, 2,6-dimethyl-4-heptanol, 2-nonanol, 3,7-dimethyl-3-octanol, ethylene glycol, propylene glycol, diethyl ether, dipropyl ether, diisopropyl ether, butyl methyl ether, butyl ethyl ether, butyl propyl ether, dibutyl ether, diisobutyl ether, tert-butyl methyl ether, tert-butyl ethyl ether, tert-butyl propyl ether, di-tert-butyl ether, dipentyl ether, diisoamyl ether, cyclopentyl methyl ether, cyclohexyl methyl ether, bromomethyl methyl ether, α,α-dichloromethyl methyl ether, chloromethyl ethyl ether, 2-chloroethyl methyl ether, 2-bromoethyl methyl ether, 2,2-dichloroethyl methyl ether, 2-chloroethyl ethyl ether, 2-bromoethyl ethyl ether, (±)-1,2-dichloroethyl ethyl ether, 2,2,2-trifluoroethyl ether, ethyl vinyl ether, butyl vinyl ether, allyl ethyl ether, allyl propyl ether, allyl butyl ether, diallyl ether, 2-methoxypropene, ethyl-1-propenyl ether, cis-1-bromo-2-ethoxyethylene, 2-chloroethyl vinyl ether, allyl-1,1,2,2-tetrafluoroethyl ether, octane, isooctane, nonane, decane, methylcyclohexane, decalin, xylene, ethylbenzene, diethylbenzene, cumeme, sec-butylbenzene, cymene, dipentene, methyl pyruvate, monomethyl ether, propylene glycol monomethyl ether, propylene glycol monoethyl ether, propylene glycol monopropyl ether, propylene glycol monomethyl ether acetate, ethyl lactate, methyl methoxypropionate, cyclopentanone, cyclohexanone, n-butyl acetate, γ-butyrolactone, diisoamyl ether, isoamyl acetate, chloroform, dichloromethane, 1,4-dioxane, hexyl alcohol, 2-heptanone, isoamyl acetate, propylene carbonate, tetrahydrofuran and the like. In one embodiment of the present disclosure, as an alcohol-based organic solvent, one or more selected from among isopropyl alcohol, propyl alcohol, butyl alcohol and hexyl alcohol are more preferred, and isopropyl alcohol is most preferred in terms of boron elution.

### Molecular Sieve

The molecular sieve (MS) according to the present disclosure functions as a dehydration means for removing moisture from an organic solvent.

As an example of the organic solvent, isopropyl alcohol may be mixed with water, and forms an azeotrope with water. When an azeotrope is formed, water and isopropyl alcohol may not be purely separated by general distillation alone, and methods other than distillation need to be used to separate water and isopropyl alcohol. However, when isopropyl alcohol is dehydrated using a molecular sieve, water and isopropyl alcohol may be separated beyond an azeotrope due to permeation selectivity of the molecular sieve.

As examples of the molecular sieve, a zeolite (0 Å to 20 Å)-based molecular sieve, a silica (mesoporous silica; 200 Å to 1000 Å)-based molecular sieve, an alumina (mesoporous alumina; 50 Å to 200 Å)-based molecular sieve and the like may be used. However, the molecular sieve is not limited thereto, and known molecular sieves may be used without particular limit as long as it has a dehydration ability to remove water from the organic solvent. Particularly, in the present disclosure, a zeolite-based molecular sieve having a pore size of 10 Å or less is suitable for removing moisture of the organic solvent.

Examples of the zeolite-based molecular sieve used to remove moisture from the organic solvent include 3 Å, 4 Å, 5 Å, 10X, 13X, 13X-AS, Cu-13X and the like by the pore size, and the pore size is preferably from 3 Å to 5 Å. Having a pore size of less than Å or greater than 5 Å is not suitable as a dehydration means since the moisture removal effect is not favorable.

When removing moisture from an organic solvent using the molecular sieve, it is difficult to obtain a high-purity organic solvent due to impurities that may be included in the molecular sieve. Examples of impurities that may be included in the molecular sieve include boron, and the impurity is mixed into the organic solvent during a dehydration process using the molecular sieve, which affects quality such as final purity of the organic solvent to be purified. Accordingly, a process of pretreating the molecular sieve to remove impurities such as boron is required. When removing moisture from an organic solvent using a molecular sieve that is not pretreated, metallic impurities such as boron may remain during an organic solvent purification process, reducing cleaning performance of a cleaning solution. Particularly, in the case of a semiconductor cleaning solution, there may be a problem of causing wafer defects.

The molecular sieve pretreatment of the present disclosure is immersing the molecular sieve in the same organic solvent as the organic solvent to be purified for a certain period of time or longer. As a method for immersing the molecular sieve in the organic solvent, mixing, stirring, solvent circulation and/or ultrasonic waves may be used as well as an act of soaking the molecular sieve in the organic solvent. When performing immersion using the mechanical means of mixing, stirring, solvent circulation and/or ultrasonic waves, removal of metallic impurities such as boron is accelerated compared to when soaking the molecular sieve in the organic solvent, resulting in a shorter immersion period. Immersing in an organic solvent different from the organic solvent to be purified is not preferred since the immersion solvent becomes an impurity to the organic solvent to be purified. In order to reduce the content of impurities, preferably boron, included in a final purified organic solvent to less than 10 ppb, preferably less than ppb and more preferably less than 1 ppb, immersion is preferably performed for a period of 50 days or longer, more preferably 140 days or longer and most preferably 180 days or longer. When performing immersion for less than the above-mentioned period, the content of metallic impurities such as boron of the molecular sieve may be 10 ppb or greater.

Such a molecular sieve may be included in a dry bed unit of the dehydration process. In particular, when a mesh is installed in any one of an inlet 201-1 and an outlet 201-2 in a dry bed unit, preferably in each of an inlet and an outlet in a dry bed unit, an inflow into the organic solvent purification process may be prevented and damage to the molecular sieve may be prevented. The mesh is preferably made of, for example, a stainless steel 304 material, but is not limited thereto. FIG. 4 is a diagram illustrating one example of a dry bed unit 111 in which a mesh is installed in an inlet and an outlet. As illustrated in FIG. 4, it is preferable to install a mesh in both an inlet 201-1 and an outlet 201-2 in a dry bed unit.

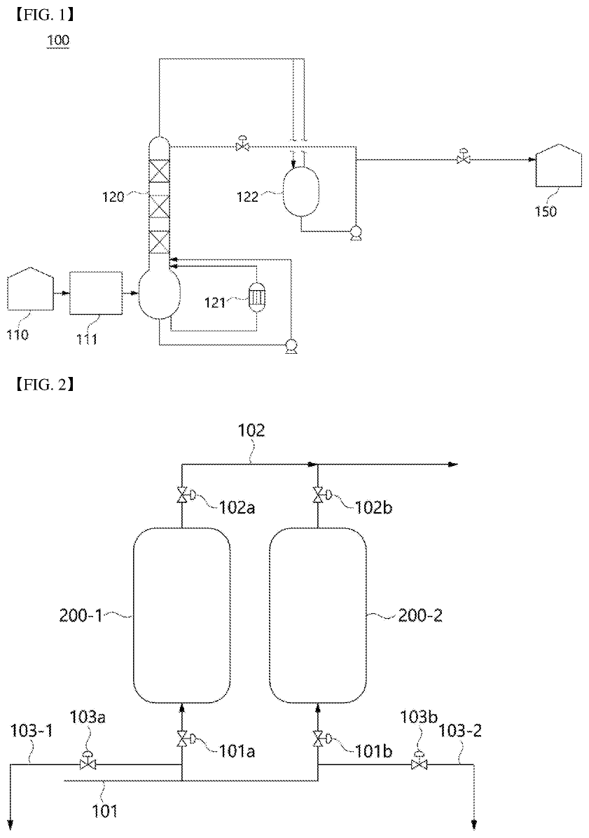

FIG. 1 illustrates a process diagram of a purification process of an organic solvent according to one embodiment of the present disclosure, and a purification tower 100 of the present disclosure includes at least one of a raw material tank 110, a dry bed unit 111, a distillation column 120, a reboiler 121, a reflux tank 122 and/or a product tank 150.

The purification process of an organic solvent according to one embodiment of the present disclosure may further include, prior to the step of removing moisture of an organic solvent using a dehydration means, performing distillation by supplying the organic solvent to a distillation column 120.

The organic solvent from which moisture is removed in the dry bed unit 111 is introduced into the distillation column 120. The step of distilling the moisture-removed organic solvent may be a step of sorting impurities using a difference in the boiling points.

The distillation column 120 may be formed as one distillation column as illustrated in FIG. 1, may also be formed with two or more distillation columns placed in parallel in order to increase purity of a final organic solvent product, and, in addition thereto, may be a multi-walled distillation column, a dividing-wall column and the like in which two or more distillations are performed in one column. However, the distillation column 120 is not limited thereto.

The organic solvent purification process of the present disclosure may further include a step of liquefying the organic solvent, which is distilled through the distillation column 120 in the step of distilling the moisture-removed organic solvent, through a condenser (not shown). Installing two or more of the condensers to be connected in parallel has a process advantage in that, even when a problem occurs with the role of the first condenser, the organic solvent may be condensed through the second condenser.

The organic solvent purification process of the present disclosure may further include, after the step of distilling the moisture-removed organic solvent, a step of re-vaporizing the liquefied impurities discharged through the lower part of the distillation column. Referring to FIG. 1, this is a step performed in a reboiler 121, and since it is difficult to completely separate impurities with one distillation, a portion of the organic solvent including impurities is returned to the distillation column 120, and vaporization and liquefaction are continuously repeated, leading to redistillation. Herein, methods known in the art may be used without limit, however, in one embodiment of the present disclosure, a heat exchange method (thermosyphon reboiler) using a density difference in vapor heat is suitable.

The organic solvent purification process of the present disclosure may further include a step of refluxing the organic solvent, which is distilled in the distillation column through the step of distilling the moisture-removed organic solvent, in a reflux tank 122. The organic solvent refluxed in the reflux tank 122 may undergo a redistillation process in the distillation column to obtain an organic solvent with higher purity. In addition, the organic solvent that has undergone repeated distillation may be sent to a product tank 150 after inspecting purity in the reflux tank 122, and therefore, a purified organic solvent with higher purity may be obtained.

### <Organic Solvent and Storage Process of Organic Solvent>

The scope of the present disclosure includes an organic solvent purified according to the organic solvent purification process of the present disclosure, and a storage process of the organic solvent. The organic solvent and the storage process of the organic solvent of the present disclosure include the description provided in the organic solvent purification process without limit.

Specifically, the final purified organic solvent according to the present disclosure may include boron in a concentration of less than 10 ppb, preferably less than 5 ppb, and more preferably less than 1 ppb.

The organic solvent storage process of the present disclosure may include a step of storing the organic solvent purified according to the process described above at a temperature of lower than 35° C., preferably at a temperature of 30° C. or lower than 30° C.

In addition, the organic solvent storage process of the present disclosure may include a step of storing the organic solvent at a temperature of lower than 35° C., preferably at a temperature of 30° C. or lower than 30° C.

The organic solvent of the present disclosure may have an acetone content of 2 ppm or less in concentration and preferably less than 1 ppm in concentration after long-term storing for 7 days or longer at a temperature of 30° C. or lower.

### <Dehydration System>

The present disclosure relates to a dehydration system for removing water from an organic solvent.

Hereinafter, preferred embodiments of the present disclosure will be described in detail with reference to drawings. However, these embodiments are presented for illustrative purposes only in order to more specifically describe the present disclosure, and it will be obvious to those skilled in the art that the scope of the present disclosure is not limited by these embodiments.

The dehydration system of the present disclosure includes a dry bed unit, and may preferably include two or more dry bed units. In addition, by a pretreatment solvent discharge unit being directly/indirectly connected to each dry bed unit, a plurality of dry bed units may serve a function for performing the molecular sieve pretreatment described above, and as necessary, may serve a function for the step of removing moisture of the organic solvent to be purified. In other words, by having a plurality of dry bed units, the molecular sieve pretreatment may be performed in another dry bed unit while performing the step of moisture removal in one dry bed unit, and as a result, a continuous purification process may be performed without a gap even during the time for molecular sieve replacement.

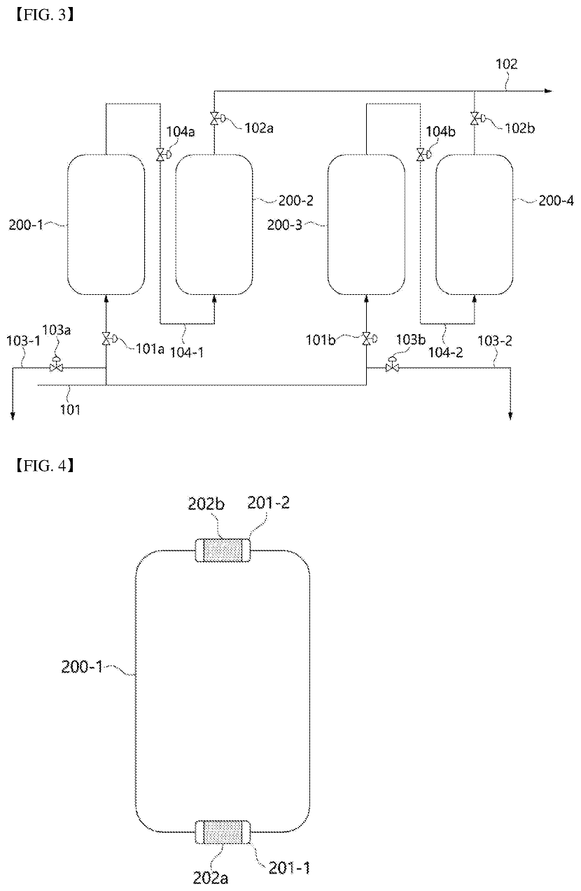

FIG. 1 is a diagram illustrating an overall process including the organic solvent purification process of one embodiment of the present disclosure, FIG. 2 specifically illustrates one example of a dry bed unit 111 of the present disclosure, and FIG. 3 illustrates another example of a dry bed unit 111 of the present disclosure.

FIG. 2 illustrates a dry bed unit 111 including two dry bed units of a first dry bed unit 200-1 and a second dry bed unit 200-2. The dehydration system of the present disclosure includes a pair of dry bed units of a first dry bed unit 200-1 and a second dry bed unit 200-2 as in FIG. 2 that are additionally connected in a series or parallel manner. FIG. 3 illustrates the dehydration system in which dry bed units are additionally connected in series.

Referring to FIG. 2, the dehydration system according to one embodiment of the present disclosure may include, as a dehydration system for removing water from an organic solvent, a first pipe unit 101, a first control valve 101a, a second control valve 101b, a second pipe unit 102, a first release valve 102a, a second release valve 102b, a first pretreatment solvent discharge unit 103-1, a second pretreatment solvent discharge unit 103-2, a first discharge valve 103a, a second discharge valve 103b, a first dry bed unit 200-1 and a second dry bed unit 200-2.

In addition, referring to FIG. 3, the dehydration system according to one embodiment of the present disclosure may include, as a dehydration system for removing water from an organic solvent, a first pipe unit 101, a first control valve 101a, a second control valve 101b, a second pipe unit 102, a first release valve 102a, a second release valve 102b, a first pretreatment solvent discharge unit 103-1, a second pretreatment solvent discharge unit 103-2, a first discharge valve 103a, a second discharge valve 103b, a third pipe unit 104-1, a fourth pipe unit 104-2, a third control valve 104a, a fourth control valve 104b, a first dry bed unit 200-1, a second dry bed unit 200-2, a third dry bed unit 200-3 and a fourth dry bed unit 200-4.

The first pipe unit 101 may be provided on at least one surface of the first dry bed unit 200-1, the second dry bed unit 200-2 and/or the third dry bed unit 200-3 to continuously supply an organic solvent under a constant flow rate.

The first control valve 101a and the second control valve 101b may be provided on at least one surface of the first pipe unit 101 to adjust the flow rate of an organic solvent supplied to each dry bed.

The second pipe unit 102 may be provided on at least one surface of the first dry bed unit 200-1, the second dry bed unit 200-2 and/or the fourth dry bed unit 200-4 to release a dehydrated organic solvent to the distillation column 120 under a constant flow rate.

The first release valve 102a and the second release valve 102b may be provided on at least one surface of the second pipe unit 102 to adjust the flow rate of a dehydrated organic solvent released from the dry bed to the distillation column.

The first pretreatment solvent discharge unit 103-1 and the second pretreatment solvent discharge unit 103-2 may be provided on at least one surface of the first pipe unit to, when pretreatment of molecular sieve is performed in the dry bed, discharge an organic solvent after the pretreatment process.

The first discharge valve 103a and the second discharge valve 103b may be provided in parallel with the first pretreatment solvent discharge unit 103-1 and the second pretreatment solvent discharge unit 103-2, respectively, to adjust the flow rate of an organic solvent discharged to the outside after the pretreatment of molecular sieve in the dry bed.

The third pipe unit 104-1 and the fourth pipe unit 104-2 may be provided on at least one surface of the first dry bed unit 200-1 and the third dry bed unit 200-3, respectively, to supply an organic solvent released from the first dry bed unit 200-1 and the third dry bed unit 200-3, respectively, to the second dry bed unit 200-2 and the fourth dry bed unit 200-4.

The third control valve 104a and the fourth control valve 104b may be provided on at least one surface of third pipe unit 104-1 and the fourth pipe unit 104-2, respectively, to adjust the flow of the released organic solvent.

The first dry bed unit 200-1, the second dry bed unit 200-2, the third dry bed unit 200-3 and the fourth dry bed unit 200-4 may be each independently driven to remove water from an organic solvent, or for pretreatment of a dehydration means.

Specifically, in FIG. 3, when at least one of the first dry bed unit 200-1 and the second dry bed unit 200-2 is driven to remove water from an organic solvent, the third dry bed unit 200-3 and the fourth dry bed unit 200-4 may be driven to pretreat a dehydration means. When a raw material has a low water content, any one of the first dry bed unit 200-1 and the second dry bed unit 200-2 may be driven to remove water from an organic solvent, and when a raw material has a high water content, the first dry bed unit 200-1 and the second dry bed unit 200-2 may be driven at the same time to remove water from an organic solvent.

By the same principle, when any one of the third dry bed unit 200-3 and the fourth dry bed unit 200-4 is driven to remove water from an organic solvent, the first dry bed unit 200-1 and the second dry bed unit 200-2 may be driven to pretreat a dehydration means.

Referring to FIG. 3, an organic solvent is supplied to the first dry bed unit 200-1 and the third dry bed unit 200-3 through the first pipe unit 101, the first dry bed unit 200-2 and the second dry bed unit 200-2 may be driven to remove water from the organic solvent, and the third dry bed unit 200-3 and the fourth dry bed unit 200-4 may be driven to pretreat a dehydration means. After that, the dehydrated organic solvent may be supplied to the distillation column 120 through the second pipe unit 102. The dehydration system of the present disclosure according to FIG. 3 has an advantage of effectively shortening the overall purification time of an organic solvent since the molecular sieve pretreatment process and the organic solvent dehydration process are continuously performed.

The present disclosure includes, in addition to the dehydration system, a purification process of an organic solvent, the process including the dehydration system.

Hereinafter, specific experimental examples are presented in order to help understand the present disclosure, however, these are for illustrative purposes only and do not limit the scope of appended claims. It will be obvious to those skilled in the art that various changes and modifications may be made for the examples within the category and the scope of technical ideas of the present disclosure, and it is also reasonable that such changes and modifications fall within the scope of the appended claims.

### EXPERIMENTAL EXAMPLE

### Experimental Example 1. Evaluation on Impurity Content in Organic Solvent Depending on Immersion Period Through Pretreatment Process

A molecular sieve was pretreated according to an embodiment of the organic solvent purification process of the present disclosure, and then moisture was removed from an organic solvent using the molecular sieve pretreated for each immersion period as a dehydration means, and the following Table 1 shows results of evaluating an impurity content in the final purified organic solvent.

Specifically, the pretreatment process was performed by immersing 850 kg of a molecular sieve in an organic solvent in a dry bed having a size of 1 m³. As the immersion solvent and the organic solvent to be purified, isopropyl alcohol was used, and as the molecular sieve, zeolite (Na₂O·Al₂O₃·2SiO₂·4.5H₂O) was used. The analysis value of boron depending on the immersion period, and the reduction rate with respect to the initial analysis value are shown in the following Table 1.

The boron analysis was performed through a direct metal analysis method using a mass spectrometer (ICP MSMS, Agilent Technologies, Inc. 8900). In the art, volatile boron was not able to be analyzed due to the analysis performed using a concentration method of boiling the sample.

TABLE 1

| Immersion Period (Days) | Analysis Value of Boron (ppb) |
|---|---|
| 0 | 30.6 |
| 1 | 28.1 |
| 7 | 25.0 |
| 10 | 18.2 |
| 15 | 11.4 |
| 50 | 1.88 |
| 180 | 0.07 |

According to the experimental data of Table 1, the boron concentration was 30.6 ppb immediately after immersing the molecular sieve in the organic solvent, and the boron concentration after 50 days was 1.88 ppb, indicating a reduction rate of about 94%. The boron concentration after 180 days was 0.07 ppb, which is less than 1 ppb, indicating a reduction rate of 96%.

It was identified that the amount of eluted boron decreased as the immersion period was extended, and the target analysis value of boron of less than 1 ppb was able to be obtained after 180 days.

### Experimental Example 2. Evaluation on Moisture Content Depending on Pore Size of Molecular Sieve

The following Table 2 shows results of an experiment for selecting a molecular sieve having suitable dehydration performance as a means for removing moisture from the organic solvent of the present disclosure. 50 g of a molecular sieve by the pore size was placed in a 1 L PFA bottle, and after filling up the bottle with an organic solvent, the bottle was shaken 10 times to induce adsorption of the molecular sieve. Isopropyl alcohol was used as the organic solvent, and zeolite (Na₂O·Al₂O₃·2SiO₂·4.5H₂O) was used as the molecular sieve. The moisture content in the organic solvent was analyzed after 7 days, and the analysis results are shown in the following Table 2.

TABLE 2

| Pore Size | 0 Å | 2 Å | 3 Å | 4 Å | 5 Å | 6 Å |
|---|---|---|---|---|---|---|
| Moisture | 24.8 | 24.0 | 16.1 | 16.5 | 16.1 | 25.0 |
| Content (ppm) | | | | | | |

According to the experimental data of Table 2, it was identified that the molecular sieve having a pore size of 3 Å to 5 Å was suitable as a dehydration means with the moisture content being reduced to 16.1 ppm to 16.5 ppm, whereas the molecular sieve having a pore size other than 3 Å to 5 Å exhibited poor effect of moisture removal, thereby being unsuitable as a dehydration means.

### Experimental Example 3. Damage to Molecular Sieve Depending on Presence of Mesh in Dry Bed Unit

The following Table 3 shows results of evaluating damage to the molecular sieve depending on the presence of a mesh in the dry bed unit in order to minimize damage to the molecular sieve used in the dehydration process. As illustrated in FIG. 4, an inlet mesh 202a and an outlet mesh 202b were respectively installed in an inlet 201-1 in the dry bed unit and an outlet 201-2 in the dry bed unit, and after filling up the first dry bed with the molecular sieve of Experimental Example 1, an organic solvent was allowed to flow. Damage to the molecular sieve depending on the presence of STS304 mesh made of stainless steel 304 is shown in the following Table 3. Isopropyl alcohol was used as the organic solvent, and zeolite (Na₂O·Al₂O₃·2SiO₂·4.5H₂O) was used as the molecular sieve. As a result of the evaluation, it was identified that, whereas the molecular sieve was partially damaged when the mesh was not installed on either one of the inlet and the outlet, the molecular sieve was not damaged when the mesh was installed on both the inlet and the outlet.

TABLE 3

| | Presence of Mesh in Dry Bed | | | |
|---|---|---|---|---|
| | Inlet Mesh Absent | Inlet Mesh Present | Inlet Mesh Absent | Inlet Mesh Present |
| | Outlet Mesh Absent | Outlet Mesh Absent | Outlet Mesh Present | Outlet Mesh Present |
| Damage to | ◯ | Δ | Δ | X |
| Molecular Sieve | | | | |

Evaluation Criteria
◯: molecular sieve damage 90% or greater
Δ: molecular sieve damage 50% or less
X: No molecular sieve damage

### Experimental Example 4. Evaluation on by-Product Generation Depending on Purified Organic Solvent Storage Temperature

The following Table 4 shows results of analyzing the amount of detected acetone for each storage temperature after long-term storing the purified organic solvent for a certain period of time in order to identify the effect of organic solvent storage temperature on the generation of by-product (acetone). The organic solvent purified according to an embodiment of the organic solvent purification process of the present disclosure was sealed and placed in a 50 ml brown glass bottle, and then the glass bottle was placed in a device where the temperature is maintained, and stored for 7 days. The amount of detected acetone is shown in the following Table 4. Isopropyl alcohol was used as the organic solvent, and ‘immediately after purification’ is a result of measuring isopropyl alcohol that has undergone the purification process immediately at room temperature (25° C.).

TABLE 4

| | Immediately | | | | | | | |
|---|---|---|---|---|---|---|---|---|
| | after | Storage Temperature (° C.) | | | | | | |
| | Purification | −5 | 10 | 20 | 30 | 35 | 40 | 50 |
| Amount of | <1 | <1 | <1 | <1 | <1 | 40 | 60 | 90 |
| Detected Acetone | | | | | | | | |
| (ppm) | | | | | | | | |

According to the experimental data of Table 4, it was identified that acetone was detected in an amount of 1 ppm or less when storing the organic solvent at a temperature of 30° C. or lower, however, acetone was detected in an amount of 40 ppm or greater at a temperature of 35° C. or higher. In particular, it was identified that, as the storage temperature increased above 35° C., the generation of acetone was facilitated, and a larger amount of acetone was detected.

In other words, it was identified that the organic solvent purified according to the present disclosure had minimized generation of acetone, a by-product, when stored at a temperature of lower than 35° C., preferably at a temperature of 30° C. or lower.

### Experimental Example 5. Evaluation on by-Product Generation Depending on Organic Solvent Storage Period

The following Table 5 shows results of analyzing the amount of detected acetone for each storage period at a certain temperature in order to identify the effect of organic solvent storage period on the generation of by-product (acetone). After filling an ISO tank lorry with an organic solvent, the organic solvent was long-term stored at a temperature of 40° C. Isopropyl alcohol was used as the organic solvent, and ‘same-day measurement’ is a result of measuring isopropyl alcohol that has undergone the purification process immediately at room temperature (25° C.). After that, the long-term stored organic solvent was collected in a 50 ml brown glass bottle, and the bottle was sealed and then moved to an analysis room to identify the amount of detected acetone for each storage period. The experimental results are shown in the following Table 5.

TABLE 5

| | Storage Period (Days) | | | | |
|---|---|---|---|---|---|
| | Same-day | | | | |
| | Measurement | 7 | 14 | 21 | 28 |
| Amount of Detected | <1 | 60.2 | 60.5 | 61.3 | 60.0 |
| Acetone (ppm) | | | | | |

According to the experimental data of Table 5, 60 ppm or more of acetone was detected from the organic solvent stored for 7 days or longer, and considering that the amount of detected acetone was maintained at a 60 ppm level even after the storage period was extended, it was identified that the amount of generated acetone tends to depend mainly on the storage temperature.

### REFERENCE NUMERAL

100: Purification Tower
110: Raw Material Tank
111: Dry Bed Unit
120: Distillation Column
121: Reboiler
122: Reflux Tank
150: Product Tank
101: First Pipe Unit
101a: First Control Valve
101b: Second Control Valve
102: Second Pipe Unit
102a: First Release Valve
102b: Second Release Valve
103-1: First Pretreatment Solvent Discharge Unit
103-2: Second Pretreatment Solvent Discharge Unit
103a: First Discharge Valve
103b: Second Discharge Valve
104-1: Third Pipe Unit
104-2: Fourth Pipe Unit
104a: Third Control Valve
104b: Fourth Control Valve
200-1: First Dry Bed Unit
200-2: Second Dry Bed Unit
200-3: Third Dry Bed Unit
200-4: Fourth Dry Bed Unit
201-1: Inlet in Dry Bed Unit
201-2: Outlet in Dry Bed Unit
202a: Inlet Mesh
202b: Outlet Mesh

1. A purification process of an organic solvent, the process comprising:
removing moisture of an organic solvent using a dehydration means; and
distilling the moisture-removed organic solvent,
wherein the dehydration means includes a pretreated molecular sieve, and the pretreatment is immersing the molecular sieve in the organic solvent.

2. The process of claim 1, wherein the organic solvent is an alcohol-based organic solvent.

3. The process of claim 1, wherein the organic solvent includes one or more selected from among isopropyl alcohol, propyl alcohol, butyl alcohol and hexyl alcohol.

4. The process of claim 1, wherein the molecular sieve is zeolite.

5. The process of claim 1, wherein the molecular sieve has a pore size of 3 Å to 5 Å.

6. The process of claim 1, wherein the pretreatment includes immersing the molecular sieve in the organic solvent for 180 days or longer.

7. The process of claim 1, wherein a content of boron included in the organic solvent purified according to the organic solvent purification process is 1 ppb or less.

8. The process of claim 1, further comprising storing the purified organic solvent at a temperature of 30° C. or lower.

9. A dehydration system for removing water from an organic solvent, the system comprising:
a first dry bed unit and a second dry bed unit;
a first pipe unit connected to each surface of the first dry bed unit and the second dry bed unit and combined into one pipe so as to introduce or release an organic solvent to the first dry bed unit and the second dry bed unit;
a second pipe unit connected to each surface of the first dry bed unit and the second dry bed unit and combined into one pipe so as to supply the organic solvent from the first dry bed unit and the second dry bed unit to a distillation column; and
a first pretreatment solvent discharge unit connected to one surface of the first pipe unit so as to discharge the organic solvent supplied to the first dry bed unit and a second pretreatment solvent discharge unit connected to one surface of the first pipe unit so as to discharge the organic solvent supplied to the second dry bed unit.

10. The system of claim 9, wherein the first pipe unit includes a first control valve capable of adjusting a flow rate of the organic solvent introduced to or released from to the first dry bed unit and a second control valve capable of adjusting a flow rate of the organic solvent introduced to or released from the second dry bed unit;
the second pipe unit includes a first release valve and a second release valve capable of adjusting a flow rate of the organic solvent supplied from the first dry bed unit and the second dry bed unit to the distillation column; and
the first pretreatment solvent discharge unit and the second pretreatment solvent discharge unit respectively include a first discharge valve and a second discharge valve capable of adjusting a flow rate of the discharged organic solvent.

11. The system of claim 9, further comprising:
a third dry bed unit and a fourth dry bed unit.

12. The system of claim 11, wherein, when any one of the first dry bed unit and the second dry bed unit is driven to remove water from the organic solvent, the third dry bed unit and the fourth dry bed unit are driven to pretreat a dehydration means.

13. A purification process of an organic solvent, the process comprising the dehydration system of claim 9.

14. A purification process of an organic solvent, the process comprising the dehydration system of claim 10.

15. A purification process of an organic solvent, the process comprising the dehydration system of claim 11.

16. A purification process of an organic solvent, the process comprising the dehydration system of claim 12.
